(19) United States
(12) Patent Application Publication
(10) Pub. No.: US 2008/0208203 A1
(43) Pub. Date: Aug. 28, 2008

(54) Bone measurement device

(75) Inventors: Marie Moindreau, Caen (FR); Loic Pinot, Bayeux (FR); Richard Eddy Field, Surrey (GB); Neil Rushton, Cambridgeshire (GB)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Benoist Girard SAS, Herouville-saint-clair Cedex (FR)

(21) Appl. No.: 12/008,396

(22) Filed: Jan. 10, 2008

(30) Foreign Application Priority Data
Feb. 26, 2007 (GB) 0703692.4

(51) Int. Cl. A61B 17/58

(52) U.S. Cl. 606/102

(57) ABSTRACT

A bone measurement device is used to measure the adequacy of the distance between a cavity in the head and neck of a femur and the outer surface of the neck of the proximal femur. A coupling element is fixedly attached to an outer surface of a femoral head. An arm extends from the coupling element and a bone measurement device having an adjustable bone contacting element thereon is coupled to the arm. An adjustable coupler is provided for moving the bone measurement device in a generally proximal-distal direction. The bone contacting element is mounted on the adjustable coupler for movement in a direction transverse to the proximal-distal direction into contact with bone when the coupling element is attached to the femoral head. The adjustable coupler for moving the bone measurement device in a proximal-distal direction and the adjustable bone contacting element are calibrated with distance markings. The markings correspond to sizes of femoral implants for implantation in the cavity.

### BACKGROUND OF THE INVENTION

This invention relates to apparatus for indicating the bone thickness between the predetermined distal end of a bone cavity located on a proximal/distal axis and the outer surface of a bone.

When preparing cavities in a bone, for example to receive the insert portion of a prosthetic component, it is necessary to drill into the bone. When fitting a prosthetic component to the proximal end of a femur when carrying out proximal epiphyseal replacement technique surgery, two proximal axially extending holes are drilled, one at an angle to the other. These holes can subsequently be joined by reaming to present a V-shaped cavity. When preparing such a cavity it is essential that one or other of the drill holes does not approach too close, or even break through, the wall of the bone. It is also common to prepare cavities, again, for example, at the end of a femur which are wide and if not accurately dimensioned, for example with regard to the width or the depth, can be too close to the wall of the bone, especially in the neck of the femur. A prosthesis for such cavities is shown in U.S. Pat. No. 6,524,343 the disclosure of which is incorporated herein by reference.

### SUMMARY OF THE INVENTION

One aspect of the present invention is to provide an apparatus which will assist in determining the thickness of the bone between the distal end of a bone cavity and the outer surface of the bone.

According to one aspect of the present invention an apparatus which will assist in determining the thickness of the bone between the distal end of a bone cavity and the outer surface of the bone.

According to one aspect of the present invention an apparatus is provided for indicating the bone thickness between the predetermined distal end of a bone cavity located on a proximal/distal axis and the outer surface. The apparatus comprises a support member provided with means for location on the bone and having adjustable distance indicating structures which can be adjusted in relation to the support member to contact the outer surface of the bone.

With this apparatus and knowing the accurate dimensions of the apparatus itself it is possible to measure the bone thickness concerned.

The apparatus may include a proximal/distal bone axis indicator which can, for example, be in the form of a guide wire. With this in place the guide wire can provide a datum which can be used to set up the apparatus.

In a preferred form the guide wire is provided by a guide pin which forms part of the location system of the support member on the bone. Thus, the apparatus can be located on this accurately placed pin.

In an alternative arrangement the location system for the support member on the bone may comprise a collar adapted to fit on the bone at a predetermined location.

The apparatus can also include an extension which can be adjusted to vary the proximal/distal length of the support member and which in one embodiment may comprise an adjustable carrier on which the adjustable distance indicator is supported.

In an alternative embodiment the extension may comprise a series of slots provided on the support member and adapted to locate on a co-operating connector carried on the location system on the bone. Use of alternate slots effectively increasing or decreasing the effective proximal/distal length of the support member.

In a preferred embodiment the adjustable distance indicator includes a sliding pin provided with distance indicating indicia i.e. score marks, which can be adjusted in relation to the support member.

The invention is not limited to operations relating to a femur and it can be used on any bone in which knowing the distance between an opening of the wall is required.

Other instruments used in this type of surgery are, for example, U.S. Patent Publication Nos. 2007/0233136 and 2007/0162038 the disclosures of which are incorporated by reference herein.

Aspects of the invention are provided by a bone measurement device used to measure the distance between a cavity in the head and neck of a femur and the outer surface of the neck of the proximal femur. The device has a coupling element for fixedly attaching it to an outer surface of a femoral head. An arm extends from the coupling element with a bone measurement device having an adjustable bone contacting element thereon coupled to the arm. An adjuster is used for moving the bone measurement device in a generally proximal-distal direction. A bone contacting element is mounted on the adjuster for movement therewith in a direction transverse to the proximal-distal direction into contact with bone when the coupling element is attached to the femoral head. The adjuster for moving the bone measurement device and the adjustable bone contacting element are calibrated with distance markings. The markings correspond to sizes of femoral implants for implantation in the cavity.

The bone measurement device for measuring the distance between a cavity in the head and neck of a femur and the neck outer surface may be mounted on a pin for insertion in a bore in a femoral head. The pin extends along a central axis of a neck of the proximal femur adjacent the femoral head. The arm is mounted on the pin. The arm extends outward of the central neck axis and distally with respect to the proximal femur. A bone measurement device mounted on the arm is moveable in a proximal-distal direction with respect to a medial surface of the femur. A bone contacting element mounted on the bone measurement device is moveable in a direction transverse to the proximal-distal direction into contact with the medial surface of the femur. The bone measurement device and the adjustable bone contacting element are calibrated with distance markings. The markings correspond to sizes of femoral implants for implantation in the cavity.

A method for determining the distance between a cavity in the neck of the femur and an outer surface of the neck includes mounting a support structure on a head of a femur. A bone measurement device is mounted on the support structure. The bone measurement device has a bone contacting element thereon. The bone measurement device is moved in a proximal distal direction based on the size of a prosthetic component to be implanted in the cavity. The bone contacting element is moved in a direction transverse to the proximal-distal direction into contact with the bone. Calibrations on the bone measurement device and the bone contacting element are read by the surgeon to determine the proper implant.

As used herein when referring to bones or other parts of the body, the term “proximal” means close to the heart and the term “distal” means more distant from the heart. The term “inferior” means toward the feet and the term “superior” means toward the head. The term “anterior” means toward the front part or the face and the term “posterior” means toward the back of the body. The term “medial” means toward the midline of the body and the term “lateral” means away from the midline of the body.

### BRIEF DESCRIPTION OF THE DRAWINGS

The invention can be performed in various ways and two embodiments will now be described by way of example and with reference to the accompanying drawings in which:

FIG. 1 is a diagrammatic side elevation of apparatus for indicating the bone thickness between the predetermined distal end of a bone cavity located on the proximal/distal axis and the outer surface of the bone according to the invention;

FIG. 2 is a diagrammatic representation of the proximal end of a femur showing how it is prepared to carry out proximal epiphyseal replacement technique surgery;

FIG. 3 is a diagrammatic cross-sectional representation of a prosthetic component for use in proximal epiphyseal replacement technique surgery and for which a prosthetic stem cavity can be prepared using the apparatus according to the invention;

FIG. 4 is a diagrammatic cross-sectional view of an alternative form of apparatus according to the invention;

FIG. 5 is an exploded isometric view of the apparatus shown in FIG. 4;

FIG. 6 is a side elevation of an alternative construction according to the invention;

FIG. 7 is an exploded view of parts of the support member;

FIG. 8 is an isometric view of the construction shown in FIG. 6; and

FIG. 9 is a cross-sectional plan view on the line IX-IX of FIG. 8.

### DETAILED DESCRIPTION

The apparatus according to the invention is particularly, although not exclusively, for use with proximal epiphyseal replacement technique surgery. An example of this type of surgery is explained and shown in U.S. Pat. No. 6,524,343 and includes resecting a femur at a position on the proximal side of its neck to locate a prosthetic femoral component which has a tapered insert portion and a proximal head portion. The insert portion is adapted for location in a prepared socket which, effectively, has inclined side to provide a tapering opening.

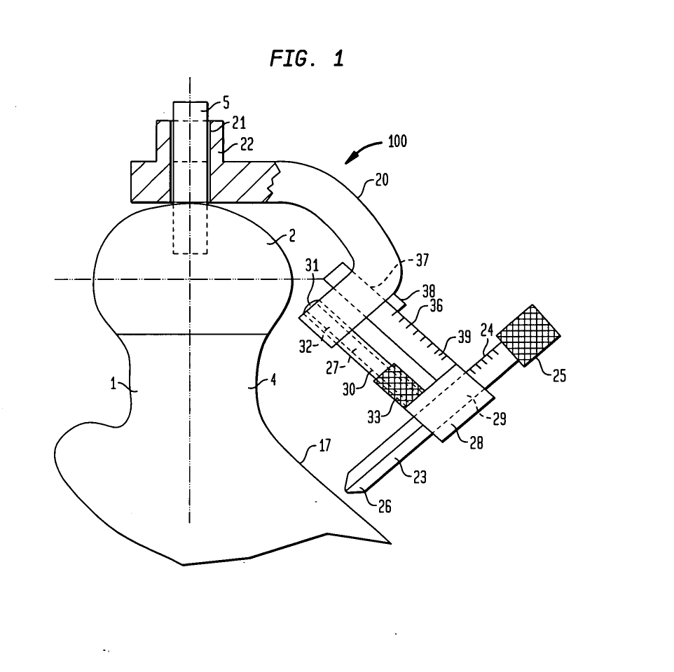

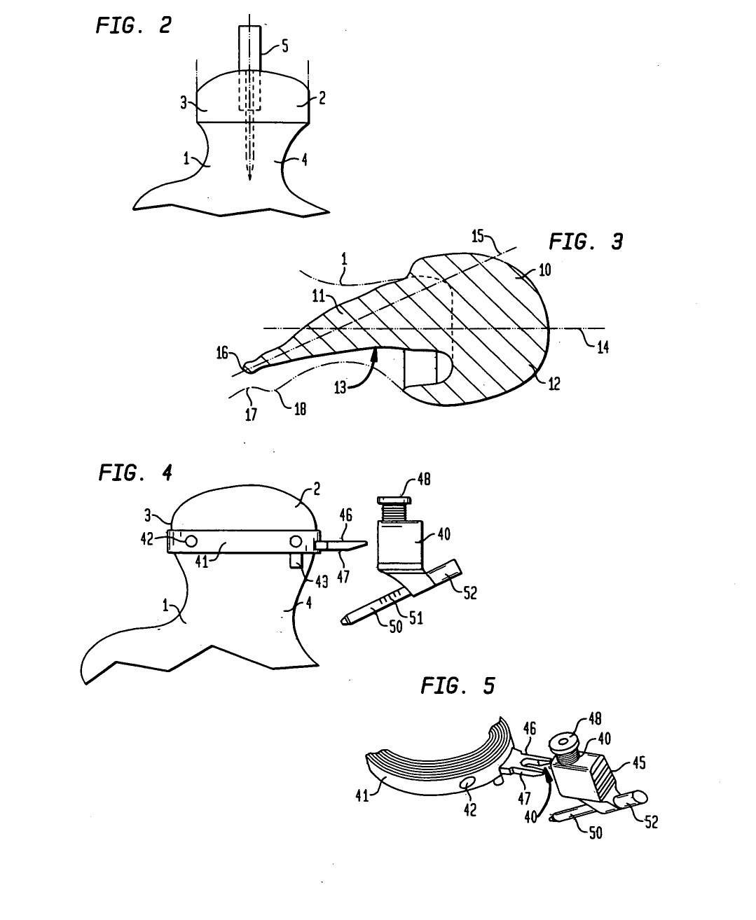

In order to carry out this type of surgery the end of a femur, indicated by reference numeral 1 in FIGS. 1 and 2, the femoral head, indicated by reference numeral 2, is first prepared by machining it with a cylindrical cutter (not shown) to provide a cylindrical portion 3. In order to accurately locate the cylindrical cutter a proximal/distal opening is drilled in the head to receive a guide wire and this opening is subsequently enlarged to receive a guide pin 5 on which the cylindrical cutter is located. This pin can also be used in the apparatus to be described herein. The neck of the femur is indicated by reference numeral 4.

FIG. 3 shows diagrammatically how a typical femoral head component for this type of surgery has a tapered insert portion 11 and a head 12 is fitted into a cavity 13 in the proximal end of the femur 1 which, in this figure, is indicated by broken lines.

In order to provide the cavity 13 a hole is drilled into the prepared femur on a axis 14 which is substantially co-axial with the proximal/distal axis of the end of the femur. A second opening is also drilled along the line of another proximal/distal axis 15 which is an angle to the line 14 to provide the basis for the tapering socket. Such a surgery is described in a U.S. patent application having the same filing date of this application entitled Apparatus For Preparing A Femur and listing Marie Mondreau, Loic Pinot, Richard Field, and Neil Rushton as inventors claiming priority to Great Britain Patent Application No. 0703691.6.

By careful measurement of the femur and knowing the dimensions of the prosthetic component it is possible to accurately determine the position of the tip 16 of the component in the bone and the present apparatus can be used for indicating the bone thickness between the predetermined distal end of the bone cavity which is located on a proximal/distal axis and the outer surface of the bone, indicated by reference numeral 17. Reference numeral 18 indicates the calcar which can be used as a reference point.

The apparatus according to the invention is shown in FIG. 1 and generally denoted as 100. The same reference numerals are used for similar parts, as indicated in FIG. 2. In FIG. 1 however apparatus 100 is being employed before the cylindrical cutter (not shown) is used to provide a cylindrical portion 3 and the guide pin 5 is used in connection with the present invention.

Apparatus 100 according to the invention comprises a support member 20 provided with means for location on the bone in the form of a prepared socket or bore 21 provided in a boss 22 which is dimensioned to be a close but sliding fit on the guide pin 5 which acts as a proximal/distal axis indicator. Thus guide pin 5 which was established on the guide wire provides a datum for setting up the apparatus. The support member 20 has an adjustable distance indicator in the form of a sliding pin 23 which is provided with distance indicating indicia 24. Sliding pin 23 can be adjusted in relation to support member 20 and carries an operating knob 25 for this purpose. As will be seen from FIG. 1 the distal end 26 of sliding pin 23 can be pushed into engagement with the medial surface 17 of the bone.

Apparatus 100 also includes an extension 27 which can be adjusted to vary the effective proximal/distal length of the support member 20. The extension 27 comprise an adjustable carrier 28 on which the sliding pin 23 is supported in a bore 29. Carrier 28 is connected to the C-shaped support member 20 by a screw threaded shaft 30. The proximal end 31 of the screw-threaded shaft 30 is rotatable in a screw-threaded bore 32 and the distal end of the shaft 30 is located in the carrier 28 but is free to rotate. The shaft has an enlarged knurled portion 33 to assist in rotating it.

Rotation of the shaft 30 causes it to progress through the screw-threaded opening 32 so that the position of the carrier 28 relative to the support member 20 can be adjusted.

The carrier 28 also has a location strut 36 which is rigidly secured thereto and extends from the carrier through an opening 37 in the support member 20 and can slide within opening 37. An indicator fin 38 is provided on the support member 20 which aligns with indicia 39 provided on the location strut 36. With this arrangement therefore the carrier 28 accurately located on the support member 20 so that its proximal/distal position can be adjusted.

With this arrangement therefore, knowing the precise dimensions of the apparatus, it can be used to measure the distance from the predetermined position of a hole to be drilled in the head to measure the thickness of bone at the predetermined position.

In most circumstances this apparatus will merely confirm that the thickness of bone is adequate but if it proves to be too small and therefore inadequate, then the surgeon will know that he cannot use the normal surgical technique to drill the holes and it may be necessary to use some different technique for applying the prosthetic insert. Thus the apparatus can prove valuable in overcoming the difficulty of knowing whether a particular technique can be used or not. If, for example, proximal epiphyseal replacement techniques surgery was intended and it was found that there was insufficient bone a different technique could be used whilst the bone was still undrilled. Initial drilling without previously measuring the bone thickness can do considerable damage to the head of the bone which could cause further difficulties if an alternative technique was subsequently to be employed.

FIGS. 4 and 5 show an alternative construction and in which similar reference numerals are used to indicate similar parts of the bone. This apparatus can conveniently be used on a head which has been prepared as shown in FIG. 2 but in this case the pin 5 need not be employed because the support member 40 is located on the bone by a collar 41. The collar can be held in place by pointed screws (not shown) being passed through screw-threaded holes 42. The collar also has a downwardly projecting pointer 43 which can be used to align collar 41 in the desire position.

In this construction support member 40 has a series of three slots 45 (as shown in FIG. 5) which are adapted to be a close sliding fit on co-operating connecting elements 46 which are in the form of a projecting lug 47 mounted on collar 41 which fits into the slots 45. A spring loaded locking pin 48 is provide which can engage an enlarged opening 49 in the lug 47 to rigidly hold the support member 40 in position. As the slots 45 are arranged in a proximal/distal direction alternative use of the slots can effectively extend or contract the length of the support member 40 in relation to the collar 41.

The distal end of support member 40 carries a sliding pin 50 which is provided with indicia 51 and has an operating knob 52. This sliding pin 50 operates in a similar manner to the sliding pin 23 in the construction shown in FIG. 1.

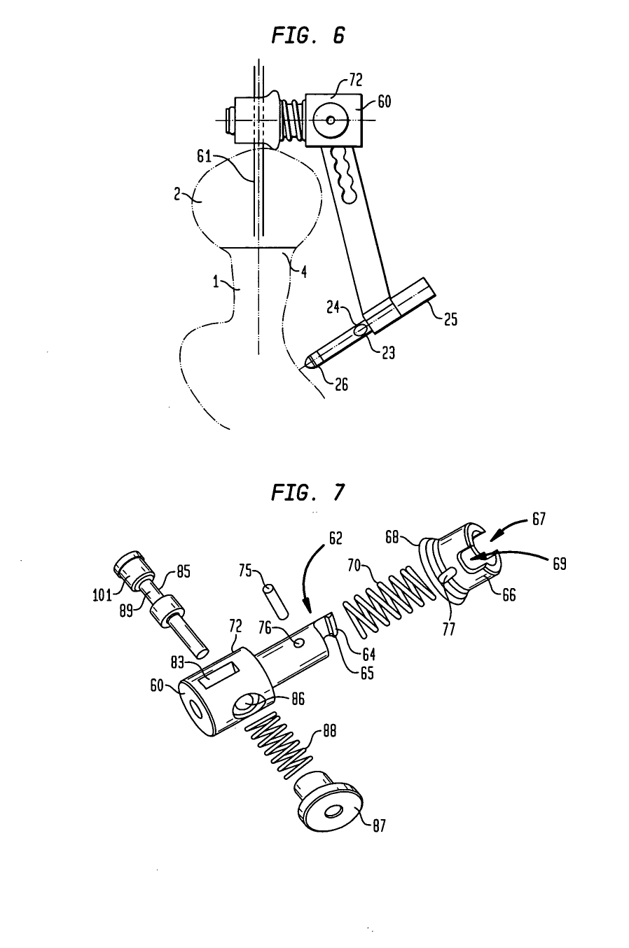

FIGS. 6, 7, 8 and 9 show another alternative construction which can be used in place of that shown in FIG. 1. In FIG. 6 the femur is indicated by broken lines and the same reference numerals are used to indicate similar parts as those shown in FIG. 1. The apparatus comprise a support member 60 provided with means for location on the bone in the form of a clamp 62 which is designed to engage and clamp on a guide wire 61.

The clamp 62 comprises a cylindrical portion 63 provided on the support member 60 and the distal end of which is provided with a claw-shaped portion 64 which has an inner curved surface 65 shaped to engage the guide wire 61. The guide wire is held in place by a clamping sleeve 66 which is bifurcated to form a shaped slot 67 at its proximal end and a flat flange 68 at its distal end.

The bore 69 of the clamping sleeve 66 is dimensioned to be a sliding fit on the cylindrical portion 63.

From FIG. 7 it will be seen that the clamping sleeve 66 is assembled over the cylindrical portion 63 with a compression coil spring 70 which also fits over the cylindrical portion 63 and is located between the flange 68 on the clamping sleeve 66 and the end of an enlarged portion 72 of the support member 60. In order to retain the clamping sleeve 66 in place a pin 75 is provided which is located in a hole 76 in the cylindrical portion 63. The pin 75 is long enough to project from one side of the hole 76 and engage at that end in a slot 77 which extends through the wall of the clamping sleeve. As the slot 77 is elongated it acts to hold the sleeve 66 in place but allows sliding movement for the length of the slot between the parts.

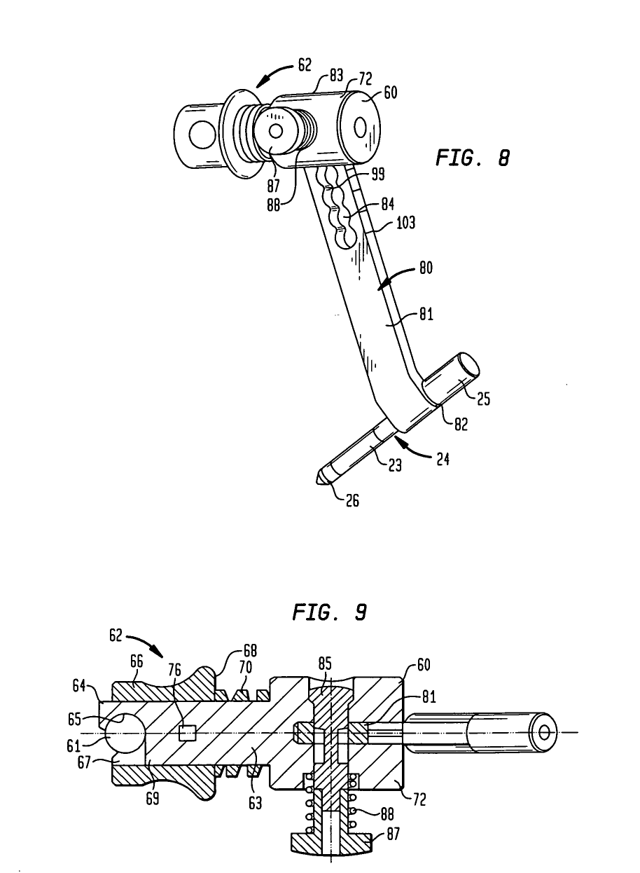

FIG. 9 shows how the shaped slot 67 in the clamping sleeve 66 extends around the claw 62 and, when a guide wire 61 is in place, acts to clamp it against the inner curved surface 65 the claw 62. To release the wire 61 it is merely necessary to move the clamping sleeve 66 against the action 62 of the spring 70 along the cylindrical portion 63 so that the wire 61 can be withdrawn from the inner curved surface 65 of the claw 64. Thus the guide wire 61 provides a datum for setting up the apparatus.

The support member 60 has an adjustable distance indicating means similar to that shown in FIG. 1 in that it includes a sliding pin 23 which is provided with distance indicating indicea 24. The sliding pin 23 can be adjusted in relation to the support member 60 and carries an operating knob 25 for this purpose. As will be seen from FIG. 6 the distal end 26 of the sliding pin 23 can be pushed into engagement with the surface 17 of the bone.

The adjustable distance indicating means is connected to the support member 60 by extension means 80 which can be adjusted to vary the proximal/distal length of the support member 60. These extension means comprise an adjustable carrier 81 on which the sliding pin 23 is supported in a bore 82. The carrier 81 is connected to the support member 60 by passing through a slot 83, best shown in FIG. 7. The carrier means 81 is in the form of a flat bar 81 and is provided with a series of linked openings 84. The bar 81 can be locked in a number of proximal/distal positions by operation of a locking pin 85 which is assembled in a bore 86 in the enlarged portion 72 of the support member 60. As will be seen from FIG. 9 the pin passes through the bore 86 and extends on the far side where it is engaged by an operating button 87. The button is screw threaded onto the end of the locking pin 85 and a compression spring 88 is located between the operating button 87 and the end of the bore 86 so that the button can be moved towards the bore thus causing the pin 85 to move with it and allow a waisted portion 89 of the pin to be aligned with the slot 72. The dimensions of the waisted portion 89 of the pin are slightly less than the gaps 99 between the openings 84 in the flat bar 81 and this enables the bar to be moved lengthwise within the slot 72 to vary its operative length. The selected length can be clamped into position by releasing the operating button 86 to allow the locking pin 85 to move into its innermost position, as shown in FIG. 9, where an enlarged portion 101 of the locking pin 85 is engaged in one of the openings 84 of the bar 81 and thus holding it in position.

Suitable indicea 103 is provided on the edge of the extension so that its precise length can be ascertained by the operator.

The apparatus is operated in the same way as that described with regard to the apparatus shown in FIG. 1.

The apparatus is not exclusively for use when carrying out proximal epiphyseal replacement technique surgery but can be used in many other operations in which a hole has to be drilled into a bone and where it is desirable to know the bone thickness between the end of the hole and the surface of the bone.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A device for measuring the distance between a cavity in the head and neck of a proximal femur and the outer surface of the neck of the proximal femur comprising:
a coupling element for fixedly attaching an outer surface of a femoral head in a defined relationship to an axis of the head;
an arm extending from the coupling element;
a bone measurement device having an adjustable bone contacting element thereon coupled to the arm and means for moving the bone measurement device in a generally proximal-distal direction, the bone contacting element mounted on the means for moving the bone measurement device for movement therewith in a direction transverse to the proximal-distal direction into contact with bone when the coupling element is attached to the femoral head.

2. The device as set forth in claim 1 wherein the means for moving the bone measurement device and the adjustable bone contacting element are calibrated with distance markings designating a distance from the axis of the head.

3. The device as set forth in claim 2 wherein the markings correspond to sizes of femoral implants for implantation in the cavity.

4. The device as set forth in claim 1 wherein the means for moving the bone measurement device in a proximal-distal direction is torque mounted on the coupling element insertion into a series of spaced slots on the arm on which the bone contacting element is coupled.

5. A device for measuring the distance between a cavity in the head and neck of a proximal femur and the outer surface of the neck of the proximal femur comprising:
a pin for insertion in a bore of a femoral head, the pin extending along a central axis of a neck of the proximal femur adjacent the femoral head;
an arm mounted on the pin, the arm extending outward of the central neck axis and distally with respect to the proximal femur;
a bone measurement device mounted on the arm and moveable in a proximal-distal direction with respect to a medial surface of the femur; and
a bone contacting element mounted on the bone measurement device moveable in a direction transverse to the proximal-distal direction into contact with the medial surface of the femur for indicating a distance from the medial surface in relation to the central axis.

6. The device as set forth in claim 4 wherein the means for moving the bone measurement device and the adjustable bone contacting element are calibrated with distance markings.

7. The device as set forth in claim 6 wherein the markings correspond to sizes of femoral implants for implantation in the cavity.

8. The device as set forth in claim 5 wherein the bone measurement device is a threaded adjustor on which the bone contacting element is mounted, the adjuster threadably engaging the arm.

9. A method for determining the distance between a cavity in the neck of the femur and an outer surface of the neck comprising:
mounting a support structure on a head of a femur;
mounting a bone measurement device on the support structure, the bone measurement device having a bone contacting element thereon;
moving the bone measurement device in a proximal distal direction a distance based on the size of a prosthetic component to be implanted in the cavity;
moving the bone contacting element in a direction transverse to the proximal-distal direction into contact with the femur; and
determining the distance between the cavity in the neck and the outer surface of the neck based on markings on the bone contacting element.

10. The method as set forth in claim 7 wherein the markings correspond to sizes of femoral implants for implantation into the cavity.

11. An apparatus for indicating the bone thickness between the predetermined distal end of a bone cavity located on a proximal/distal axis and the outer surface of the bone comprising a support member provided with means for location on the bone defining an axis and having adjustable distance indicating means which can be adjusted in relation to the support means axis to contact the outer surface of the bone to indicate a distance in relation to the support means axis.

12. The apparatus as claimed in claim 11 which includes a proximal/distal bone axis indicator.

13. The apparatus as claimed in claim 12 in which said proximal/distal bone axis indicator is provided by a guide wire.

14. The apparatus as claimed in claim 13 in which said guide wire is provided by a guide pin which forms part of the means for location of the support member on the bone.

15. The apparatus as claimed in claim 11 in which the means for location of the support member on the bone comprises a collar adapted to fit on the bone at a predetermined location.

16. The apparatus as claimed in claim 11 including extension means which can be adjusted to vary the proximal/distal length of the support member.

17. The apparatus as claimed in claim 16 in which the extension means comprises an adjustable carrier on which the adjustable distance indicating means is supported.

18. The apparatus as claimed in claim 16 in which the extension means comprises a series of slots provided in said adjustable carrier adapted to locate on co-operating connection means carried on said means for location on the bone.

19. The apparatus as claimed in claim 11 in which the adjustable distance indicating means include a sliding pin provided with distance indicating indicea which can be adjusted in relation to the support member.
